(19) United States
(12) Patent Application Publication
(10) Pub. No.: US 2026/0169007 A1
(43) Pub. Date: Jun. 18, 2026

(54) AN IMPROVED ASSAY METHOD FOR DETERMINING THE POTENCY OF RECOMBINANT PROTEIN

(71) Applicant: Kashiv BioSciences, LLC, Piscataway, NJ (US)

(72) Inventors: Shalini Sharma, Piscataway, NJ (US); Praveen Kumar Anidil Kizhakinagath, Ahmedabad (IN); Kavya Siddhartha Jash, Ahmedabad (IN)

(21) Appl. No.: 18/720,887

(22) PCT Filed: Dec. 17, 2022
(86) PCT No.: PCT/IB2022/062427

§ 371 Date: Jun. 17, 2024

(30) Foreign Application Priority Data
Dec. 17, 2021 (IN) 202121058990

(51) Int. Cl. G01N 33/68; G01N 33/50; G01N 33/53

(52) U.S. Cl. CPC G01N 33/6869; G01N 33/505; G01N 33/53; G01N 33/6854; G01N 2333/55; G01N 2333/70503

(57) ABSTRACT

The present invention provides an improved assay method for determining the potency of recombinant proteins by culturing the immune cells such as antigen presenting cell and/or T cell in culture media comprising FBS less than 10%. Furthermore, the invention provides a method to eliminate or reduce the false estimation of potency of target recombinant protein. The assay method determines the potency of recombinant protein by evaluating the expression of inflammatory protein. More specifically the present invention describes the IL-2 inhibition assays for determination of potency of CTLA4 IgG1.

### FIELD OF INVENTION

The present invention provides an improved assay method for determining the potency of recombinant proteins by culturing the immune cells such as antigen presenting cell &/or T cell in culture media comprising FBS less than 10%. Furthermore, the invention provides a method to eliminate or reduce the false estimation of potency of target recombinant protein. The assay method determines the potency of recombinant protein by evaluating the expression of inflammatory protein. More specifically the present invention describes the IL-2 inhibition assays for determination of potency of CTLA4 IgG1.

### BACKGROUND OF INVENTION

Every area of the biomedical sciences is in need of a system to assay chemical and biochemical reactions and determine the presence and quantity of particular analyte. Numerous methodologies have been developed over the years to meet the demands of these fields. The approach for assessing the potency of biological products is to develop a biological assay (bioassay) that measures the activity of the product.

The bioassay is essential to report on the product's potency, by providing an assessment of the molecule's biological activity. Selection of an appropriate bioassay method, excipients, and cell line has its challenges because bioassays can be difficult to develop new assay method by changing different new parameters. Besides, one of the major challenges is to develop the bioassay which eliminates or reduce the false determination of potency of targeted protein. The applicant noticed that existing assay are not suitable for full curve analysis of protein formulations which contains sugars and/or surfactant which affect the osmolality of the cell & thereby produce false result. The problem further compounded when the targeted protein is formulated in two formulation liquid and lyophilized formulation. If liquid formulation has sugar while the lyophilized formulation does not have sugar, it would be difficult to obtain desirable 90% CI. This false estimation of potency may lead to rejecting of desired compound and further it would be difficult to justify to regulatory bodies. The present invention solved the existing problem by providing an improved assay method of recombinant proteins for an example CTLA4-IgG1 by culturing the immune cells such as antigen presenting cell &/or T cell in culture media comprising FBS less than 10% and provides desired relative potency by measuring expression of inflammatory protein such as IL-2 protein which correspond to inhibitory potential of CTLA4-IgG1.

### SUMMARY OF INVENTION

In an embodiment the present invention is to describe the inflammatory protein inhibition assays for determination of potency of recombinant protein. The assay method is provided for the detection of inhibitory activity of inhibitory protein wherein the immune cells such as antigen presenting cell and/or T cell are cultured in culture media comprising FBS less than 10% In an embodiment the present invention provides an improved assay method for determining potency and an improved 95% confidence intervals of recombinant protein in different formulations by modulating the amount of Fetal bovine serum in the assay method.

In an embodiment, the present invention provides improve suitability and comparability of protein mixture formulated in prefilled syringe (PFS) and vial antigen presenting cells and/or T cells in Fetal bovine serum less than 10% provides improved 95% confidence intervals.

In an embodiment the present invention provides an improved assay method for determining potency of recombinant protein comprising; the antigen presenting cells and/or T cells are added in multi well plate separately or simultaneously.

In certain embodiment the antigen presenting cells and/or T cells are seeded in multi well plate together and thereafter targeted recombinant protein is incorporated. In certain embodiment the antigen presenting cells is seeded in multi well plate and thereafter targeted recombinant protein is incorporated.

In an embodiment the present invention provides process for reducing false potency estimation of recombinant protein; wherein cultured antigen presenting cells and/or T cells in Fetal bovine serum less than 10% provides improved potency compared to antigen presenting cells and/or T cells cultured in 10% Fetal bovine serum.

In an embodiment, the present invention provides an improved assay method for determining potency of recombinant protein comprising;
a) culturing suitable antigen presenting cells and/or T cells in media comprising Fetal bovine serum less than 10%;
b) incubating treated antigen presenting cells and/or T cells for suitable time and suitable temperature;
c) mixing suitable recombinant protein with suitable stimulants to stimulate T cells in the cultured antigen presenting cells and/or T cells;
d) incubating treated mixture for suitable time and suitable temperature;
e) adding reagent in the incubated multi well plate;
f) determining the inhibition of inflammatory protein;
   wherein cultured antigen presenting cells and/or T cells in Fetal bovine serum less than 10% provides improved potency compared to antigen presenting cells and/or T cells cultured in 10% Fetal bovine serum.

In an embodiment, the present invention provides an improved assay method for determining potency of recombinant protein comprising;
a) casting of suitable antigen presenting cells in multi well plate;
b) adding suitable recombinant protein in the multi well plate;
c) incubating multi well plate for suitable time and suitable temperature;
d) incorporating the suitable T cells into multi well plate;
e) adding suitable stimulant into treated mixture of multi well plate for stimulation of T cells;
f) incubating multi well of step (e) for suitable time and suitable temperature; g) adding reagent in the incubated multi well plate;
h) determining the inhibition of inflammatory protein;
   wherein cultured antigen presenting cells and/or T cells in Fetal bovine serum less than 10% provides improved potency compared to antigen presenting cells and/or T cells cultured in 10% Fetal bovine serum.

In an embodiment, the present invention provides an improved assay method provides 95% confidence intervals of recombinant protein comprising;
a) culturing suitable antigen presenting cells and/or T cells in media comprising Fetal bovine serum less than 10%;
b) incubating treated antigen presenting cells and/or T cells for suitable time and suitable temperature;
c) mixing suitable recombinant protein with suitable stimulants to stimulate T cells in the cultured antigen presenting cells and/or T cells;
d) incubating treated mixture for suitable time and suitable temperature;
e) adding reagent in the incubated multi well plate;
f) determining the inhibition of inflammatory protein;
   wherein cultured antigen presenting cells and/or T cells in Fetal bovine serum less than 10% provides improved 95% confidence intervals compared to antigen presenting cells and/or T cells cultured in 10% Fetal bovine serum.

In an embodiment, the present invention provides a process for reducing false potency estimation of recombinant protein comprising;
a) culturing suitable antigen presenting cells and/or T cells in media comprising Fetal bovine serum less than 10%;
b) incubating treated antigen presenting cells and/or T cells for suitable time and suitable temperature;
c) mixing suitable recombinant protein with suitable stimulant to stimulate T cells in the cultured antigen presenting cells and/or T cells;
d) incubating treated mixture for suitable time and suitable temperature;
e) adding reagent in the incubated multi well plate;
f) determining the inhibition of inflammatory protein;
   wherein cultured antigen presenting cells and/or T cells in Fetal bovine serum less than 10% provides improved potency compared to antigen presenting cells and/or T cells cultured in 10% Fetal bovine serum.

### BRIEF DESCRIPTION OF THE FIGURES

FIG. 1: Representative data for inhibition assay

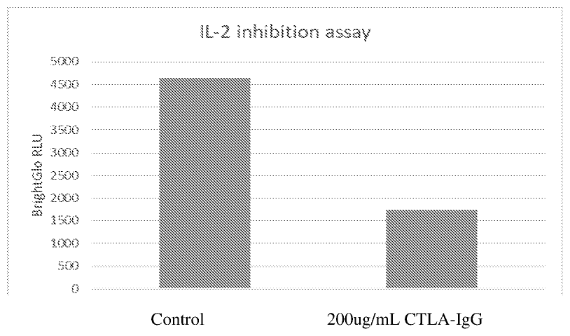

FIG. 2: Anti-CD3 antibody as stimulants in IL-2 inhibition assay

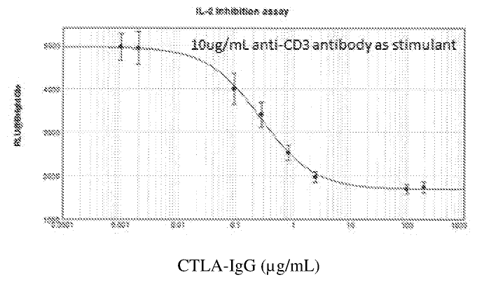

FIG. 3: PHA as stimulants in IL-2 inhibition assay

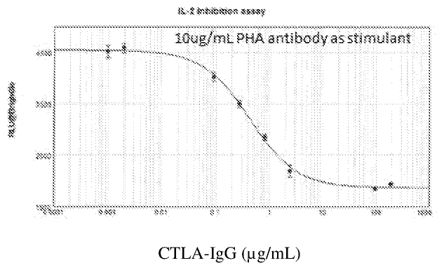

FIG. 4: Vial compared to vial in IL-2 inhibition assay

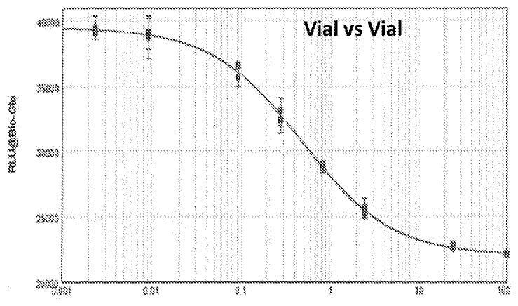

FIG. 5: PFS compared to vial in IL-2 inhibition assay

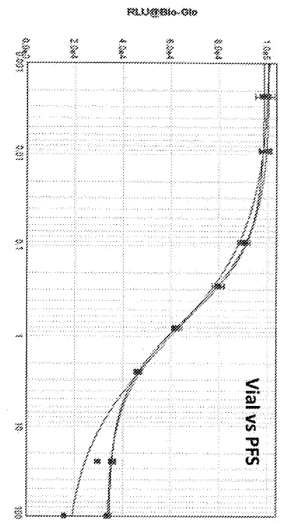

FIG. 6: Improved assay performances of IL-2 inhibition assay with reduced FBS percentage.

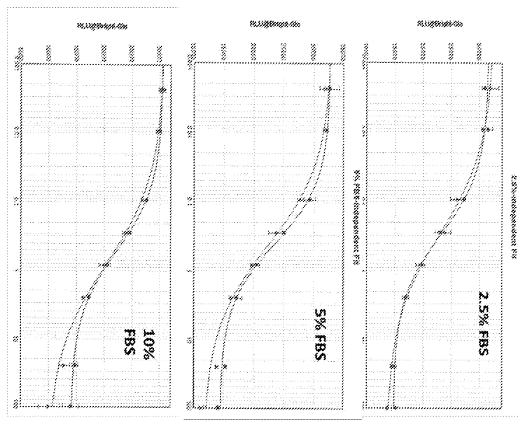

### DETAILED DESCRIPTION OF INVENTION

The term “recombinant protein” refers to IgG1 antibody or fragment thereof and fusion protein produced by recombinant technology known in the art. The recombinant protein is selected from IgG1, IgG2, IgG3, IgG4 and fusion proteins.

The term IgG1 antibody is selected from Abatacept, Rituximab, Palivizumab, Infliximab, Trastuzumab, Alemtuzumab, Adalimumab, Ibritumomab, Omalizumab, Cetuximab, Bevacizumab, Natalizumab, Eculizumab, Certolizumab pegol, Ustekinumab, Canakinumab, Golimumab, Ofatumumab, Tocilizumab, Denosumab, Belimumab, Ipilimumab, Brentuximab vedotin, Pertuzumab, Trastuzumab emtansine, Raxibacumab, Obinutuzumab, Siltuximab.

The term “fusion protein” refers to as CTLA4-Ig molecules can include, for example, CTLA4-Ig proteins in monomer, dimer, trimer, tetramer, pentamer, hexamer, or other multimeric forms. CTLA4-Ig molecules can comprise a protein fusion with at least an extracellular domain of CTLA4 and an immunoglobulin constant region. CTLA4-Ig molecules can have wild-type or mutant sequences, for example, with respect to the CTLA4 extracellular domain and immunoglobulin constant region sequences. CTLA4-Ig monomers, alone, or in dimer, tetramer or other multimer form, can be glycosylated. The proteins created through the joining of two or more genes that originally coded for separate proteins. A CTLA4-Ig molecule is also capable of binding to CD80 and/or CD86. In certain embodiment, the CTLA4-Ig is Abatacept.

The fusion proteins are made using recombinant DNA techniques. Fusion protein consisting of receptor including but not limited to selected from CTLA4, TNFR, VEGF, HER-2, PCSK9 fused with constant region of immunoglobulin selected from IgG1, IgG2, IgG3 and IgG4. In addition, any modification is performed in natural amino acid to obtain desired biological activity.

The term “Abatacept” refers to a recombinant DNA generated fusion protein used to treat the symptoms of rheumatoid arthritis and to prevent joint damage caused by these conditions. Abatacept is a biological product developed for immunosuppression by blocking T cell activation through inhibition of costimulatory signals and is indicated for treatment of rheumatoid arthritis. Abatacept is a soluble homodimeric fusion protein of two identical subunits covalently linked by one disulfide bond. Each subunit consists of the modified amino acid sequence of the human cytotoxic lymphocyte associated antigen 4 (CTLA4), human immunoglobin IgG1 hinge, CH2 and CH3 region (Fc). Modification to the original sequences were introduced to avoid unintended disulfide bond formation and to reduce the ability of complement activation.

The term “antigen presenting cells” or “APC cells” refers to cells capable to express B7 receptors or ligands (CD80 and CD86); for an example “antigen presenting cells” are selected from B cells, dendritic cells, Raji cells.

The term “Raji cells” refers to as cells endogenously expressing the B7 ligands.

The term “T cells” or “CD28 Effector Cells” refers to as expressing endogenous TCR/CD3 antibody and CD28, and a luciferase reporter driven by TCR/CD3 and CD28 pathway-dependent response elements; for an example “T cells” is IL-2 Luc Jurkat E6.1 cell.

The term “inflammatory protein” refers to as cytokines are selected from IL 2, IL4, IL7, IL9, IL15, IL21, GM-CSF, TNF-alpha, IL-6, IFN-gamma and IL-17A.

The term “IL-2 Protein” is interchangeable with Interleukin 2.

The term “Potency” is interchangeable with relative potency.

The term “False potency estimation” refers that at the lab scale, 95% confidence intervals accurately identify the difference between potential and less potential molecules. For this reason, in the assay method, improper molecule may be picked up as a potential molecule due to the misguidance of 95% confidence intervals and relative potency.

The term “PFS” refers to as prefilled syringe formation or liquid formulation of recombinant protein.

The term “vial” refers to as lyophilized formulation of recombinant protein.

The term “Suitability” refers to assay is capable to be used for protein mixture formulated in lyophilized and liquid formulation.

The term “Comparability” refers to protein mixture formulated in liquid and lyophilized formulation wherein liquid formulation presence of sugar and lyophilized formulation either doesn't have sugar or sugar is present in less or no amount than liquid formulation. Therefore, due to variation of sugar concentration in different presentation (PFS and vial) of the same protein may create difference in assay result (difference in 95% CI) which may lead to false potency estimation.

The term “sugar” refers to monosaccharides, disaccharides, and polysaccharides. Examples of sugars include, but are not limited to, sucrose, trehalose, dextrose, and others.

The term “Luminescence signal” refers to as the emission of light by a substance as a result of a chemical reaction (chemiluminescence) or an enzymatic reaction (bioluminescence).

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

The term “comprises” or “comprising” is used in the present description, it does not exclude other elements or steps. For the purpose of the present invention, the term “consisting of” is considered to be an optional embodiment of the term “comprising of”. If hereinafter a group is defined to comprise at least a certain number of embodiments, this is also to be understood to disclose a group which optionally consists only of these embodiments.

In an embodiment the present invention is to describe the inflammatory protein inhibition assays for determination of potency of recombinant protein. The assay method is provided for the detection of inhibitory activity of inhibitory protein wherein the immune cells such as antigen presenting cell and/or T cell are cultured in culture media comprising FBS less than 10% In an embodiment the present invention provides an improved assay method for determining potency and an improved 95% confidence intervals of recombinant protein in different formulations by modulating the amount of Fetal bovine serum in the assay method.

In an embodiment, the present invention provides improve suitability and comparability of protein mixture formulated in prefilled syringe (PFS) and vial antigen presenting cells and/or T cells in Fetal bovine serum less than 10% provides improved 95% confidence intervals.

In an embodiment, the present invention provides an improved assay method for determining potency of recombinant protein comprising; the antigen presenting cells and/or T cells are added in multi well plate separately or simultaneously.

In certain embodiment, the antigen presenting cells and/or T cells are seeded in multi well plate together and thereafter targeted recombinant protein is incorporated. In certain embodiment the antigen presenting cells is seeded in multi well plate and thereafter targeted recombinant protein is incorporated.

In an embodiment, the present invention provides process for reducing false potency estimation of recombinant protein; wherein cultured antigen presenting cells and/or T cells in Fetal bovine serum less than 10% provides improved potency compared to antigen presenting cells and/or T cells cultured in 10% Fetal bovine serum.

In an embodiment, the present invention provides an improved assay method of recombinant protein comprising; the cultured antigen presenting cells and/or T cells in Fetal bovine serum less than 10% provides improved 95% confidence intervals difference is less than 100.

In an embodiment, the present invention provides an improved assay method of recombinant protein comprising; 95% confidence intervals difference is less than 100, thereby reduces the incident of false potency estimation.

In an embodiment, the present invention also addresses the problem of using excipients which affect the osmolarity of cells wherein the use of sugars and surfactants affect the cell adversely which may lead to undesired result in assay and thereby skilled person will obtain false result.

In an embodiment, the present invention provides an improved assay method for determining potency of recombinant protein comprising;
a) culturing suitable antigen presenting cells and/or T cells in media comprising Fetal bovine serum less than 10%;
b) incubating treated antigen presenting cells and/or T cells for suitable time and suitable temperature;
c) mixing suitable recombinant protein with suitable stimulants to stimulate T cells in the cultured antigen presenting cells and/or T cells;
d) incubating treated mixture for suitable time and suitable temperature;
e) adding reagent in the incubated multi well plate;
f) determining the inhibition of inflammatory protein;
   wherein cultured antigen presenting cells and/or T cells in Fetal bovine serum less than 10% provides improved potency compared to antigen presenting cells and/or T cells cultured in 10% Fetal bovine serum.

In an embodiment, the present invention provides improve suitability and comparability of protein mixture formulated in liquid prefilled syringe (PFS) and lyophilized vial.

In an embodiment, the present invention provides an improved assay method for determining potency of recombinant protein wherein suitable antigen presenting cell is a Raji cell and suitable T cell is an IL-2 Luc Jurkat E6.1 cell.

In an embodiment, the present invention provides an improved assay method for determining potency of recombinant protein wherein recombinant protein is fusion protein.

In an embodiment, the present invention provides an improved assay method for determining potency of recombinant protein wherein fusion protein is CTLA4 IgG1.

In an embodiment, the present invention provides an improved assay method for determining potency of recombinant protein wherein CTLA4 IgG1 is Abatacept.

In an embodiment, the present invention provides an improved assay method for determining potency of recombinant protein wherein suitable the stimulant is selected from Phytohemagglutinin (PHA) or anti-CD3 antibody.

In an embodiment, the present invention provides an improved assay method for determining potency of recombinant protein wherein reagent is Bright-Glo reagent.

In an embodiment, the present invention provides an improved assay method for determining potency of recombinant protein wherein the incubation of treated antigen presenting cells and/or T cells is performed at least for about 15 minutes; preferably for about 15 minutes to about 60 minutes at about 37° C. with 5% Co₂.

In an embodiment, the present invention provides an improved assay method for determining potency of recombinant protein wherein the incubation of treated antigen presenting cells and/or T cells is performed for about 30 minutes to about 60 minutes at about 37° C. with 5% CO_{2.}

In an embodiment, the present invention provides an improved assay method for determining potency of recombinant protein wherein the incubation of treated mixture of step (d) is performed at least for about 2 hours; preferably for about 2 hours to about 8 hours at about 37° C. with 5% CO_{2.}

In an embodiment, the present invention provides an improved assay method for determining potency of recombinant protein wherein the incubation of treated mixture of step (d) is performed for about 5 hours to about 6 hours at about 37° C. with 5% CO_{2.}

In an embodiment, the present invention provides an improved assay method for determining potency of recombinant protein wherein the antigen presenting cells and/or T cells are added in multi well plate separately or simultaneously.

In an embodiment, the present invention provides an improved assay method of recombinant protein comprising; the cultured antigen presenting cells and/or T cells in Fetal bovine serum less than 10% provides improved potency by improving 95% confidence intervals of inflammatory protein inhibition.

In an embodiment, the present invention provides a process for reducing false potency estimation of recombinant protein comprising;
a) culturing suitable antigen presenting cells and/or T cells in media comprising Fetal bovine serum less than 10%;
b) incubating treated antigen presenting cells and/or T cells for suitable time and suitable temperature;
c) mixing suitable recombinant protein with suitable stimulant to stimulate T cells in the cultured antigen presenting cells and/or T cells;
d) incubating treated mixture for suitable time and suitable temperature;
e) adding reagent in the incubated multi well plate;
f) determining the inhibition of inflammatory protein;
   wherein cultured antigen presenting cells and/or T cells in Fetal bovine serum less than 10% provides improved potency compared to antigen presenting cells and/or T cells cultured in 10% Fetal bovine serum.

In an embodiment, the present invention provides an improved assay method for determining potency of recombinant protein comprising;
a) culturing suitable Raji cells and/or IL-2 Luc Jurkat E6.1 cells in media comprising Fetal bovine serum less than 10%;
b) incubating treated Raji cells and/or IL-2 Luc Jurkat E6.1 cells at least for about 15 minutes; preferably for about 15 minutes to about 60 minutes at about 37° C. with 5% CO₂;
c) mixing suitable CTLA4 IgG1 with Phytohemagglutinin (PHA) or anti-CD3 antibody to stimulate T cells in the cultured Raji cells and/or IL-2 Luc Jurkat E6.1 cells;
d) incubating treated mixture at least for about 2 hours; preferably for about 2 hours to about 8 hours at about 37° C. with 5% CO_{2;}
e) adding Bright-Glo reagent in the incubated multi well plate;
f) determining the inhibition of inflammatory protein;
   wherein cultured Raji cells and/or IL-2 Luc Jurkat E6.1 cells in Fetal bovine serum less than 10% provides improved potency compared to Raji cells and/or IL-2 Luc Jurkat E6.1 cells cultured in 10% Fetal bovine serum.

In an embodiment, the present invention provides an improved assay method for determining potency of recombinant protein comprising;
a) culturing suitable Raji cells and/or IL-2 Luc Jurkat E6.1 cells in media comprising Fetal bovine serum less than 10%;
b) incubating treated Raji cells and/or IL-2 Luc Jurkat E6.1 cells for about 30 minutes to about 60 minutes at about 37° C. with 5% CO_{2;}
c) mixing suitable Abatacept with Phytohemagglutinin (PHA) or anti-CD3 antibody to stimulate T cells in the cultured Raji cells and/or IL-2 Luc Jurkat E6.1 cells;
d) incubating treated mixture at least for about 5 hours to about 6 hours at about 37° C. with 5% CO_{2;}
e) adding Bright-Glo reagent in the incubated multi well plate;
f) determining the inhibition of IL-2 protein;
wherein cultured Raji cells and/or IL-2 Luc Jurkat E6.1 cells in Fetal bovine serum less than 10% provides improved potency compared to Raji cells and/or IL-2 Luc Jurkat E6.1 cells cultured in 10% Fetal bovine serum.

In an embodiment, the present invention provides an improved assay method for determining potency of recombinant protein comprising;
a) casting of suitable antigen presenting cells in multi well plate;
b) adding suitable recombinant protein in the multi well plate;
c) incubating multi well plate for suitable time and suitable temperature;
d) incorporating the suitable T cells into multi well plate;
e) adding suitable stimulant into treated mixture of multi well plate for stimulation of T cells;
f) incubating multi well of step (e) for suitable time and suitable temperature;
g) adding reagent in the incubated multi well plate;
h) determining the inhibition of inflammatory protein;
   wherein cultured antigen presenting cells and/or T cells in Fetal bovine serum less than 10% provides improved potency compared to antigen presenting cells and/or T cells cultured in 10% Fetal bovine serum.

In an embodiment, the present invention provides an improved assay method for determining potency of recombinant protein comprising;
a) casting of suitable Raji cells in multi well plate;
b) adding suitable CTLA4 IgG1 antibody in the multi well plate;
c) incubating multi well plate at least for about 15 minutes; preferably for about 15 minutes to about 60 minutes at about 37° C. with 5% CO_{2;}
d) incorporating the IL-2 Luc Jurkat E6.1 cells into multi well plate;
e) adding Phytohemagglutinin (PHA) or anti-CD3 antibody into treated mixture of multi well plate for stimulation of T cells;
f) incubating multi well of step (e) for at least for about 2 hours; preferably for about 2 hours to about 8 hours at about 37° C. with 5% CO_{2;}
g) adding Bright-Glo reagent in the incubated multi well plate;
h) determining the inhibition of inflammatory protein;
   wherein cultured Raji cells and/or IL-2 Luc Jurkat E6.1 cells in Fetal bovine serum less than 10% provides improved potency compared to Raji cells and/or IL-2 Luc Jurkat E6.1 cells cultured in 10% Fetal bovine serum.

In an embodiment, the present invention provides an improved assay method for determining potency of recombinant protein comprising;
a) casting of suitable Raji cells in multi well plate;
b) adding suitable CTLA4 IgG1 antibody in the multi well plate;
c) incubating multi well plate for about 30 minutes to about 60 minutes at about 37° C. with 5% CO_{2;}
d) incorporating the IL-2 Luc Jurkat E6.1 cells into multi well plate;
e) adding Phytohemagglutinin (PHA) or anti-CD3 antibody into treated mixture of multi well plate for stimulation of T cells;
f) incubating multi well of step (e) for about 5 hours to about 6 hours at about 37° C. with 5% CO_{2;}
g) adding Bright-Glo reagent in the incubated multi well plate;
h) determining the inhibition of IL-2 protein;
   wherein cultured Raji cells and/or IL-2 Luc Jurkat E6.1 cells in Fetal bovine serum less than 10% provides improved potency compared to Raji cells and/or IL-2 Luc Jurkat E6.1 cells cultured in 10% Fetal bovine serum.

In an embodiment, the present invention provides an improved assay method provides 95% confidence intervals of recombinant protein comprising;
a) culturing suitable antigen presenting cells and/or T cells in media comprising Fetal bovine serum less than 10%;
b) incubating treated antigen presenting cells and/or T cells for suitable time and suitable temperature;
c) mixing suitable recombinant protein with suitable stimulants to stimulate T cells in the cultured antigen presenting cells and/or T cells;
d) incubating treated mixture for suitable time and suitable temperature;
e) adding reagent in the incubated multi well plate;
f) determining the inhibition of inflammatory protein;
   wherein cultured antigen presenting cells and/or T cells in Fetal bovine serum less than 10% provides improved 95% confidence intervals compared to antigen presenting cells and/or T cells cultured in 10% Fetal bovine serum.

In an embodiment, the present invention provides an improved assay method provides 95% confidence intervals of recombinant protein comprising;
a) culturing suitable Raji cells and/or IL-2 Luc Jurkat E6.1 cells in media comprising Fetal bovine serum less than 10%;
b) incubating treated Raji cells and/or IL-2 Luc Jurkat E6.1 cells at least for about 15 minutes; preferably for about 15 minutes to about 60 minutes at about 37° C. with 5% CO₂;
c) mixing suitable CTLA4 IgG1 with Phytohemagglutinin (PHA) or anti-CD3 antibody to stimulate T cells in the cultured Raji cells and/or IL-2 Luc Jurkat E6.1 cells;
d) incubating treated mixture at least for about 2 hours; preferably for about 2 hours to about 8 hours at about 37° C. with 5% CO₂;
e) adding Bright-Glo reagent in the incubated multi well plate;
f) determining the inhibition of inflammatory protein;
   wherein cultured Raji cells and/or IL-2 Luc Jurkat E6.1 cells in Fetal bovine serum less than 10% provides improved 95% confidence intervals compared to antigen presenting cells and/or T cells cultured in 10% Fetal bovine serum.

In an embodiment, the present invention provides an improved assay method provides 95% confidence intervals of recombinant protein comprising;
a) culturing suitable Raji cells and/or IL-2 Luc Jurkat E6.1 cells in media comprising Fetal bovine serum less than 10%;
b) incubating treated Raji cells and/or IL-2 Luc Jurkat E6.1 cells for about 30 minutes to about 60 minutes at about 37° C. with 5% CO_{2;}
c) mixing suitable Abatacept with Phytohemagglutinin (PHA) or anti-CD3 antibody to stimulate T cells in the cultured Raji cells and/or IL-2 Luc Jurkat E6.1 cells;
d) incubating treated mixture at least for about 5 hours to about 6 hours at about 37° C. with 5% CO_{2;}
e) adding Bright-Glo reagent in the incubated multi well plate;
f) determining the inhibition of IL-2 protein;
   wherein cultured Raji cells and/or IL-2 Luc Jurkat E6.1 cells in Fetal bovine serum less than 10% provides improved 95% confidence intervals compared to antigen presenting cells and/or T cells cultured in 10% Fetal bovine serum.

In an embodiment, the present invention provides a process for reducing false potency estimation of recombinant protein comprising;
a) culturing suitable antigen presenting cells and/or T cells in media comprising Fetal bovine serum less than 10%;
b) incubating treated antigen presenting cells and/or T cells for suitable time and suitable temperature;
c) mixing suitable recombinant protein protein with suitable stimulant to stimulate T cells in the cultured antigen presenting cells and/or T cells;
d) incubating treated mixture for suitable time and suitable temperature;
e) adding reagent in the incubated multi well plate;
f) determining the inhibition of inflammatory protein;
   wherein cultured antigen presenting cells and/or T cells in Fetal bovine serum less than 10% provides improved potency compared to antigen presenting cells and/or T cells cultured in 10% Fetal bovine serum.

In an embodiment, the present invention provides a process for reducing false potency estimation of recombinant protein comprising;
a) culturing suitable Raji cells and/or IL-2 Luc Jurkat E6.1 cells in media comprising Fetal bovine serum less than 10%;
b) incubating treated Raji cells and/or IL-2 Luc Jurkat E6.1 cells at least for about 15 minutes; preferably for about 15 minutes to about 60 minutes at about 37° C. with 5% CO₂;
c) mixing suitable CTLA4 IgG1 with Phytohemagglutinin (PHA) or anti-CD3 antibody to stimulate T cells in the cultured Raji cells and/or IL-2 Luc Jurkat E6.1 cells;
d) incubating treated mixture at least for about 2 hours; preferably for about 2 hours to about 8 hours at about 37° C. with 5% CO_{2;}
e) adding Bright-Glo reagent in the incubated multi well plate;
f) determining the inhibition of inflammatory protein;
   wherein cultured Raji cells and/or IL-2 Luc Jurkat E6.1 cells in Fetal bovine serum less than 10% provides improved potency compared to Raji cells and/or IL-2 Luc Jurkat E6.1 cells cultured in 10% Fetal bovine serum.

In an embodiment, the present invention provides a process for reducing false potency estimation of recombinant protein comprising;
a) culturing suitable Raji cells and/or IL-2 Luc Jurkat E6.1 cells in media comprising Fetal bovine serum less than 10%;
b) incubating treated Raji cells and/or IL-2 Luc Jurkat E6.1 cells for about 30 minutes to about 60 minutes at about 37° C. with 5% CO_{2;}
c) mixing suitable Abatacept with Phytohemagglutinin (PHA) or anti-CD3 antibody to stimulate T cells in the cultured Raji cells and/or IL-2 Luc Jurkat E6.1 cells; d) incubating treated mixture at least for about 5 hours to about 6 hours at about 37° C. with 5% CO_{2;}
e) adding Bright-Glo reagent in the incubated multi well plate;
f) determining the inhibition of IL-2 protein;
   wherein cultured Raji cells and/or IL-2 Luc Jurkat E6.1 cells in Fetal bovine serum less than 10% provides improved potency compared to Raji cells and/or IL-2 Luc Jurkat E6.1 cells cultured in 10% Fetal bovine serum.

In an embodiment, the invention provides the suitable assay which remove or minimize false potency estimation for protein formulated in liquid PFS and lyophilized vial.

In an embodiment, the CTLA4 IgG1 is Abatacept.

In an embodiment, the invention provides the concentrations of CTLA4 IgG1 in assay plate are selected from about 100 μg/ml to about 0.00100 μg/ml.

In an embodiment, the invention provides the concentrations of CTLA4 IgG1 in assay plate are 50 μg/ml, 25 μg/ml, 2 μg/ml, 0.666 μg/ml, 0.222 μg/ml, 0.074 μg/ml, 0.00592 μg/ml, 0.00148 μg/ml.

In an embodiment, the invention provides the IL-2 Protein refers to Cytokine Protein or IL-2 Luc Jurkat cells.

In an embodiment, the invention provides the suitable antigen-presenting cells is a Raji cells.

In an embodiment, the Raji cells are 4 million cells/mL and concentration is selected from about 10 μL/well to about 40 μL/well.

In an embodiment, the Raji cells are 4 million cells/mL and concentration is 25 μL/well.

In an embodiment, the invention provides the suitable T cells is an IL-2 Luc Jurkat E6.1 cells.

In an embodiment, the IL-2 Luc Jurkat E6.1 cells are 8 million cells/mL and concentration is selected from about 10 μL/well to about 40 μL/well.

In an embodiment, the IL-2 Luc Jurkat E6.1 cells are 8 million cells/mL with concentration is 25 μL/well.

In an embodiment, the invention provides the stimulant is Phytohemagglutinin (PHA) or anti-CD3 antibody.

In an embodiment, the invention provides the stimulant is act as primary stimulant of T cells.

In an embodiment, the invention provides the concentration of stimulant is selected from 5 μg/ml to 20 μg/ml.

In an embodiment, the invention provides the concentration of stimulant is 10 μg/ml.

In an embodiment, the invention provides the volume of stimulant is selected from about 10 μL to about 35 μL.

In an embodiment, the invention provides the volume of stimulant is 25 μL.

In an embodiment, the invention provides the detection reagent is Bright-Glo reagent.

In an embodiment, the invention provides the concentration of detection reagent is selected from about 50 μL to about 150 μL.

In an embodiment, the invention provides the concentration of detection reagent is 100 μL.

In an embodiment, the invention provides the detection reagent is added at end of incubation.

In an embodiment, the invention provides the detection reagent is added after removing assay plate from the incubator.

In an embodiment, the invention provides shaking the assay plate for about 5 minutes to about 10 minutes.

In an embodiment, the invention provides the detection reagent is added for measuring the luminescence signal of sample containing assay plate.

In an embodiment, the invention provides the detection reagent is added for measuring the luminescence signal of sample containing assay plate using multimode plate reader.

In an embodiment of present invention, the use of Fetal bovine serum in the assay, wherein the concentration of Fetal bovine serum is selected from about 1% FBS to about 15% FBS.

In an embodiment of present invention, the use of Fetal bovine serum in the assay, wherein the concentration of Fetal bovine serum is selected from about 2.5% FBS, about 5% FBS and about 10% FBS.

In an embodiment, the invention provides the assay plates are kept at about 37° C. and 5% CO₂.

In certain embodiment of the invention, the multi well plate refers to more than 1 well plate.

In certain embodiment of the invention, the multi well plate or assay plate is selected from 96 well plate, 384 well plate.

In an embodiment, the invention provides the incubation time for assay plate which is considered as preincubation is at least for about 15 minutes.

In an embodiment, the invention provides the incubation time for assay plate which is considered as preincubation is selected from about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes.

In an embodiment, the invention provides the incubation time for assay plate which is considered as post incubation is at least for about 2 hours.

In an embodiment, the invention provides the incubation time for assay plate which is considered as post incubation is selected from about 2 hours, about 3 hours, about 4 hours, about 5 hours, about 6 hours, about 7 hours, about 8 hours.

### Solutions Preparation:

In an embodiment, the invention provides the complete RPMI for IL-2 Luc Jurkat Cells comprising mixing of 500 ml RPMI-1620, 55 ml FBS, 5.5 ml PenStrep and 11 ml Geneticin.

In an embodiment, the invention provides the complete RPMI for Raji Cells comprising mixing of 500 ml RPMI-1620, 55 ml FBS, 5.5 ml PenStrep.

In an embodiment, the invention provides the assay media composition comprising mixing of 490 ml RPMI-1620, 10 ml FBS, 5.5 ml PenStrep.

In an embodiment, the invention provides the drug dilution media composition comprising mixing of 39.84 mL assay media and 0.16 mL anti-CD3 antibody or Phytohemagglutinin (PHA) (1 mg/mL).

In an embodiment, the invention provides the Bright-Glo reagent composition comprising 1 vial Bright-Glo Luciferase Assay Substrate and 100 mL Bright-Glo Luciferase Assay Buffer.

The present invention provides an example for illustrative purpose and scope should not be considered limiting the examples.

### Example 1: To Derive DRC of CTLA4 IgG1 with Anti-CD3 Antibody and PHA as Stimulants in IL-2 Inhibition Assay

Raji cells (4 million cells/mL; 25 μL/well) cells is seeded to a white 96 well plate containing CTLA4 IgG1 samples (25 μL). The plate is incubated for 20-30 minutes at 37° C. and 5% CO2. IL-2 Luc Jurkat E6.1 cells are added to the assay plate (8 million cells/mL; 25 μL/well), followed by 25 μL of 10 μg/mL Phytohemagglutinin (PHA) or anti-CD3 antibody. The assay plate is incubated at 37° C. and 5% CO2 for 5-6 hours post incubation, 100 μL Bright-Glo reagent is added luminescence signal was measured using a multimode plate reader. All the dilution are prepared in 0-5% FBS containing assay media.

### Example 2: To Derive DRC of CTLA4 IgG1 with Anti-CD3 Antibody and PHA as Stimulants in IL-2 Inhibition Assay

Mixture of Raji cells (4 million cells/mL) and IL-2 Luc Jurkat E6.1 cells cells (8 million cells/mL) is seeded to a white 96 well plate and incubated for 30-60 min at 37° C. and 5% CO2. CTLA4 IgG1 prepared in 25 μL of 10 μg/mL PHA or anti-CD3 antibody containing media is added to the cells (50 μL). The plate is incubated for 5 h-6 h at 37° C. and 5% CO2 followed by addition of 100 μL Bright-Glo reagent and luminescence signal measured using a multimode plate reader. All the dilution are prepared in 0-5% FBS containing assay media.

### Observation:

Both anti CD3 antibody and PHA induced stimulation of T cells and Raji cells were inhibited by CTLA4 IgG.

TABLE 1
EC50 value of PHA and anti-CD3 antibody

| Stimulants | EC50 (μg/mL) |
|---|---|
| PHA | 0.429 |
| Anti CD3 antibody | 0.271 |

### Example 3: To Test Varying FBS Concentration in IL-2 Inhibition Assay

Serially diluted CTLA4 IgG1 is added to the assay plate along with the controls, followed by addition of Raji cells. The plate is incubated for 37° C. and 5% CO₂ for 30 min. Next, IL-2 Luc Jurkat cells are added to the predefined wells, followed by the stimulant PHA. Assay is performed in assay media containing 2.5%, 5% and 10% FBS. The plate is incubated for 6 hours at 37° C. and 5% CO₂. After 6 hours, Bright-Glo reagent is added, and luminescence measured.

TABLE 2
Analysis of potency of PFS against vial in different
percentage of FBS assay conditions

| Sample Name | Assay condition | Potency | 95% CI |
|---|---|---|---|
| Potency of PFS | 2.5% FBS | 106.4 | 72.8/127.2 |
| using vial as | 5% FBS | 138.6 | 62.1/138 |
| refence standard | 10% FBS | 132.6 | 48.3/151.6 |
| Potency of Vial | 2.5% FBS | 106.6 | 81.4/118.6 |
| using vial as | | | |
| refence standard | | | |

### Observation:

Evaluation using different serum percentage demonstrates that the vial and PFS samples are comparable at low concentration of serum when compared to 10%, 95% CI of the potency values are best found to be in the range of 70-130% in 2.5% FBS.

1. An improved assay method for determining potency of recombinant protein comprising;
a) culturing suitable antigen presenting cells and/or T cells in media comprising Fetal bovine serum less than 10%;
b) incubating treated antigen presenting cells and/or T cells for suitable time and suitable temperature;
c) mixing suitable recombinant protein with suitable stimulant to stimulate T cells in the cultured antigen presenting cells and/or T cells;
d) incubating treated mixture for suitable time and suitable temperature;
e) adding reagent in the incubated multi well plate;
f) determining the inhibition of inflammatory protein;
wherein cultured antigen presenting cells and/or T cells in Fetal bovine serum less than 10% provides improved potency compared to antigen presenting cells and/or T cells cultured in 10% Fetal bovine serum.

2. The assay as claimed in claim 1, further improve suitability and comparability of protein mixture formulated in liquid prefilled syringe (PFS) and lyophilized vial.

3. The improved assay method as claimed in claim 1, wherein suitable antigen presenting cell is a Raji cell and suitable T cell is an IL-2 Luc Jurkat E6.1 cell.

4. The improved assay method as claimed in claim 1, wherein recombinant protein is fusion protein.

5. The improved assay method as claimed in claim 4, wherein fusion protein is CTLA4 IgG1.

6. The improved assay method as claimed in claim 5, wherein CTLA4 IgG1 is Abatacept.

7. The improved assay method as claimed in claim 1, wherein the suitable stimulant is selected from Phytohemagglutinin (PHA) or anti-CD3 antibody.

8. The improved assay method as claimed in claim 1, wherein reagent is Bright-Glo reagent.

9. The improved assay method as claimed in claim 1, wherein the incubation of treated antigen presenting cells and/or T cells is performed at least for about 15 minutes; preferably for about 15 minutes to about 60 minutes at about 37° C. with 5% CO₂.

10. The improved assay method as claimed in claim 9, wherein the incubation of treated antigen presenting cells and/or T cells is performed for about 30 minutes to about 60 minutes at about 37° C. with 5% CO₂.

11. The improved assay method as claimed in claim 1, wherein the incubation of treated mixture of step (d) is performed at least for about 2 hours; preferably for about 2 hours to about 8 hours at about 37° C. with 5% CO_{2.}

12. The improved assay method as claimed in claim 11, wherein the incubation of treated mixture of step (d) is performed for about 5 hours to about 6 hours at about 37° C. with 5% CO_{2.}

13. The improved assay method as claimed in claim 1, wherein the antigen presenting cells and/or T cells are added in multi well plate separately or simultaneously.

14. An improved assay method of recombinant protein comprising; the cultured antigen presenting cells and/or T cells in Fetal bovine serum less than 10% provides improved potency by improving 95% confidence intervals of inflammatory protein inhibition.

15. A process for reducing false potency estimation of recombinant protein comprising;
a) culturing suitable antigen presenting cells and/or T cells in media comprising Fetal bovine serum less than 10%;
b) incubating treated antigen presenting cells and/or T cells for suitable time and suitable temperature;
c) mixing suitable recombinant protein with suitable stimulant to stimulate T cells in the cultured antigen presenting cells and/or T cells;
d) incubating treated mixture for suitable time and suitable temperature;
e) adding reagent in the incubated multi well plate;
f) determining the inhibition of inflammatory protein;
wherein cultured antigen presenting cells and/or T cells in Fetal bovine serum less than 10% provides improved potency compared to antigen presenting cells and/or T cells cultured in 10% Fetal bovine serum.

16. The process for reducing false potency estimation as claimed in claim 15, wherein suitable antigen presenting cell is a Raji cell and suitable T cell is an IL-2 Luc Jurkat E6.1 cell.

17. The process for reducing false potency estimation as claimed in claim 15, wherein recombinant protein is Abatacept.

18. The process for reducing false potency estimation as claimed in claim 15, wherein the suitable stimulant is selected from Phytohemagglutinin (PHA) or anti-CD3 antibody.

19. The process for reducing false potency estimation as claimed in claim 15, wherein reagent is Bright-Glo reagent.

20. The process for reducing false potency estimation as claimed in claim 15, wherein the incubation of treated antigen presenting cells and/or T cells is performed for about 30 minutes to about 60 minutes at about 37° C. with 5% CO_{2.}

21. The process for reducing false potency estimation as claimed in claim 15, wherein the incubation of treated mixture of step (d) is performed for about 5 hours to about 6 hours at about 37° C. with 5% CO₂.
